(19) United States
(12) Patent Application Publication
(10) Pub. No.: US 2025/0161250 A1
(43) Pub. Date: May 22, 2025

(54) COMPOSITIONS AND METHODS FOR TREATMENT OF IDIOPATHIC PULMONARY FIBROSIS

(71) Applicant: NUFORMIX TECHNOLOGIES LIMITED, London (GB)

(72) Inventors: Joanne HOLLAND, Cambridgeshire (GB); Lee BORTHWICK, Newcastle upon Tyne (GB)

(21) Appl. No.: 18/841,734

(22) PCT Filed: Feb. 28, 2022
(86) PCT No.: PCT/IB2022/000089

§ 371 Date: Aug. 27, 2024

(51) Int. Cl. A61K 31/196; A61K 31/4418; A61K 31/496; A61P 11/00

(52) U.S. Cl. CPC A61K 31/196; A61K 31/4418; A61K 31/496; A61P 11/00

(57) ABSTRACT

The treatment of idiopathic pulmonary fibrosis (IPF) using a therapeutically effective amount of a combination of tranilast and pirfenidone, or a therapeutically effective amount of a combination of tranilast and nintedanib, is described. Tranilast may be administered as an adjunctive therapy to the treatment of IPF by either pirfenidone or nintedanib. The combination of tranilast and pirfenidone and the combination of tranilast and nintedanib may also be synergistically effective to treat IPF. Methods and pharmaceutical compositions for treating idiopathic pulmonary fibrosis (IPF) are also described.

### STATEMENT REGARDING PRIOR DISCLOSURE UNDER 37 C.F.R. 1.77(b)(6)

A press release providing a general mention of the pre-clinical study in Example 1 was made on Mar. 2, 2021. Pursuant to the U.S. Patent and Trademark guidance of 78 Fed. Reg. 11076 (Feb. 14, 2013), Applicant is identifying this disclosure in lieu of filing a declaration under 37 C.F.R. 1.130(a). Applicant believes that such disclosure is subject to the exceptions of 35 U.S.C. 102(b)(1)(A) or 35 U.S.C. 102(2)(b)(A) as having been made or having originated from one or more members of the inventive entity of this invention.

### TECHNICAL FIELD

The invention relates to the treatment of idiopathic pulmonary fibrosis (IPF) using a therapeutically effective amount of a combination of tranilast and pirfenidone, or a therapeutically effective amount of a combination of tranilast and nintedanib. The invention also relates to methods, uses, and pharmaceutical compositions to treat IPF using a therapeutically effective amount of a combination of tranilast and pirfenidone, or a therapeutically effective amount of a combination of tranilast and nintedanib.

### BACKGROUND

Interstitial lung disease (ILD) damages a person's lung by impairing the lung's ability to absorb oxygen. ILD is characterized by varying amounts of inflammation, scarring, or both. Of the over 200 types of ILD idiopathic pulmonary fibrosis (IPF), which has no known cause, is the most common. A chronic, progressive disease, IPF exhibits fibrosis build up over time, increasing breathlessness and a patient's need for and dependence on oxygen. Common signs and symptoms of IPF include shortness of breath and a persistent dry, hacking cough. Someone suffering from IPF may have a loss of appetite and gradual weight loss and in some cases clubbing (widening and rounding) of fingers and toes due to the lack of oxygen. Typically affecting people between the ages of 50-70 the progression of IPF varies from person to person. In some patient's IPF progression is steady while in others IPF progresses by sudden declines in lung function. For these reasons it is difficult to forecast how long a person diagnosed with IPF will live, though a prognosis of 3-5 years after diagnosis. At this time there are only two therapeutics used to treat IPF.

One therapeutic used to treat IPF is pirfenidone. Pirfenidone slows IPF progression by reducing lung fibrosis downregulating the production of growth factors and procollagens I and II. Having antifibrotic, anti-inflammatory, and antioxidant effects, pirfenidone, sold under the Esbriet tradename, is orally administered as a capsule or tablet. Side effects identified for pirfenidone include skin discoloration and redness, itching, skin rash, sunburn, and stomach upset, pain, or discomfort. The US FDA Orange Books lists the following US patents for the 801 mg tablet formulation of pirfenidone: U.S. Pat. Nos. 7,566,729, 7,635,707, 7,767,700, 7,816,383, 7,910,610, 8,013,002, 8,084,475, 8,318,780, US83815, U.S. Pat. Nos. 8,420,674, 8,592,462, 8,609,701, 8,648,098, 8,754,109, 8,778,947, 9,561,217, and U.S. Ser. No. 10/188,637; the disclosures of which are incorporated herein by reference.

The other IPF therapeutic is nintedanib, which is administered as nintedanib esylate under the OFEV tradename. Administered orally as a capsule nintedanib inhibits both nonreceptor tyrosine kinases (nRTKs) and receptor tyrosine kinases (RTKs). Nintedanib's therapeutic efficacy in IPF is based on its ability to inhibit of the NRTKs platelet-derived growth factor receptor (PDGFR) α and β; fibroblast growth factor receptor (FGFR) 1, 2, and 3; vascular endothelial growth factor receptor (VEGFR) 1, 2, and 3, which increase fibroblast proliferation, migration, and transformation. Vomiting, abdominal pain, and diarrhea have been identified as side effects with nintedanib. The US FDA Orange Book lists the following US patents for nintedanib: U.S. Pat. Nos. 6,762,180, 7,119,093, 9,907,756, U.S. Ser. No. 10/105,323, and U.S. Ser. No. 10/154,990; the disclosures of which are incorporated herein by reference.

Even with the therapeutics pirfenidone and nintedanib as the accepted standard of care there is a need for additional therapeutics to treat IPF. There is also a need to avoid or reduce the side effects experienced under the current standard of care using pirfenidone or nintedanib to treat IPF. This invention addresses such needs by combining pirfenidone or nintedanib with tranilast to treat IPF and according to some embodiments of the invention reducing the pirfenidone and nintedanib doses required under the current standard of care to achieve clinical benefit.

### SUMMARY OF THE INVENTION

The invention relates to a method of treating idiopathic pulmonary fibrosis (IPF) by administering to a patient in need thereof a combined amount of tranilast and pirfenidone therapeutically effective to treat idiopathic pulmonary fibrosis (IPF). The invention relates to the use of tranilast and pirfenidone in a combined amount for therapeutically effective treatment of idiopathic pulmonary fibrosis (IPF). Tranilast may be administered as an adjunctive therapy administered with pirfenidone under the standard of care. In a method of treatment or a use of the invention the tranilast and pirfenidone may be administered in a combined amount synergistically effective to treat IPF. The tranilast and pirfenidone may be administered in the same or different pharmaceutical compositions or routes of administration. For example, tranilast may be administered as an inhaled pharmaceutical composition and pirfenidone orally administered or may be orally administered in the same or separate pills or capsules.

The invention also relates to a pharmaceutical composition comprising, consisting essentially of, or consisting of tranilast and pirfenidone, in a combined amount, and a pharmaceutically acceptable excipient for therapeutically effective treatment of idiopathic pulmonary fibrosis (IPF). In a pharmaceutical composition of the invention the tranilast and pirfenidone may be present in a combined amount synergistically effective to treat IPF.

The invention also relates to a method of treating idiopathic pulmonary fibrosis (IPF) by administering to a patient in need thereof a combined amount of tranilast and nintedanib therapeutically effective to treat idiopathic pulmonary fibrosis (IPF). The invention relates to the use of a combined amount of tranilast and nintedanib therapeutically effective to treat idiopathic pulmonary fibrosis (IPF). Tranilast may be administered as an adjunctive therapy administered with nintedanib under the standard of care. In a method of treatment or a use of the invention the tranilast and nintedanib may be administered in a combined amount synergistically effective to treat IPF. The tranilast and nintedanib may be administered in the same or different pharmaceutical compositions or routes of administration. For example, tranilast may be administered as an inhaled pharmaceutical composition and nintedanib orally administered or may be orally administered in the same or separate pills or capsules.

The invention relates to a pharmaceutical composition comprising, consisting essentially of, or consisting of tranilast and nintedanib, in a combined amount, and a pharmaceutically acceptable excipient for therapeutically effective treatment of idiopathic pulmonary fibrosis (IPF). In a pharmaceutical composition of the invention the tranilast and nintedanib may be present in a combined amount synergistically effective to treat IPF.

### BRIEF DESCRIPTION OF THE FIGURES

FIG. 1a depicts the graphs showing secreted Fibrotic marker levels in PCLuS culture supernatants (144 hrs only—% change from baseline, pirfenidone).

FIG. 1b depicts the graphs showing secreted inflammatory marker levels in PCLuS culture supernatants (144 hrs only—% change from baseline, pirfenidone).

FIG. 2a depicts the graphs showing secreted Fibrotic marker levels in PCLuS culture supernatants (144 hrs only—% change from baseline, nintedanib).

FIG. 2b depicts the graphs showing secreted inflammatory marker levels in PCLuS culture supernatants (144 hrs only—% change from baseline, nintedanib).

FIG. 3a depicts the graphs showing secreted MCP-1 levels in PCLS culture supernatants (Single Treatments—Raw values).

FIG. 3b depicts the graphs showing secreted MCP-1 levels in PCLS culture supernatants (Single Treatments—% change from baseline).

FIG. 3c depicts the graphs showing secreted MCP-1 levels in PCLS culture supernatants (Tranilast (31.25 μM) with pirfenidone Treatments only—Raw values).

FIG. 3d depicts the graphs showing secreted MCP-1 levels in PCLS culture supernatants (Tranilast (125 μM) with pirfenidone Treatments only—Raw values).

FIG. 3e depicts the graphs showing secreted MCP-1 levels in PCLS culture supernatants (Tranilast (31.25 μM) with pirfenidone Treatments only—% change from baseline).

FIG. 3f depicts the graphs showing secreted MCP-1 levels in PCLS culture supernatants (Tranilast (125 μM) with pirfenidone Treatments only—% change from baseline).

FIG. 3g depicts the graphs showing secreted MCP-1 levels in PCLS culture supernatants (Tranilast (31.25 μM) with nintedanib Treatments only—Raw values).

FIG. 3h depicts the graphs showing secreted MCP-1 levels in PCLS culture supernatants (Tranilast (125 μM) with nintedanib Treatments only—Raw values).

FIG. 3i depicts the graphs showing secreted MCP-1 levels in PCLS culture supernatants (Tranilast (31.25 μM) with nintedanib Treatments only—% change from baseline).

FIG. 3j depicts the graphs showing secreted MCP-1 levels in PCLS culture supernatants (Tranilast (125 μM) with nintedanib Treatments only—% change from baseline).

FIG. 4a shows a comparison of secreted MCP-1 levels in PCLS culture supernatants after 4-day dosing of tranilast (TL) and pirfenidone (Pir) alone and in combination.

FIG. 4b shows a comparison of secreted MCP-1 levels in PCLS culture supernatants after 4-day dosing of tranilast (TL) and nintedanib (Nin) alone and in combination.

FIG. 5 shows MCP-1 secretions as a percent of baseline at T=144 hours (6 days) as a mean of 6 slices.

FIG. 6a depicts the graphs showing secreted Collagen 1a1 levels in PCLS culture supernatants (Single Treatments—Raw values).

FIG. 6b depicts the graphs showing secreted Collagen 1a1 levels in PCLS culture supernatants (Single Treatments—% change from baseline)

FIG. 6c depicts the graphs showing secreted Collagen 1a1 levels in PCLS culture supernatants (Tranilast (31.25 μM) with pirfenidone Treatments only—Raw values).

FIG. 6d depicts the graphs showing secreted Collagen 1a1 levels in PCLS culture supernatants (Tranilast (125 μM) with pirfenidone Treatments only—Raw values).

FIG. 6e depicts the graphs showing secreted Collagen 1a1 levels in PCLS culture supernatants (Tranilast (31.25 μM) with pirfenidone Treatments only—% change from baseline).

FIG. 6f depicts the graphs showing secreted Collagen 1a1 levels in PCLS culture supernatants (Tranilast (125 μM) with pirfenidone Treatments only—% change from baseline).

FIG. 6g depicts the graphs showing secreted Collagen 1a1 levels in PCLS culture supernatants (Tranilast (31.25 μM) with nintedanib Treatments only—Raw values).

FIG. 6h depicts the graphs showing secreted Collagen 1a1 levels in PCLS culture supernatants (Tranilast (125 μM) with nintedanib Treatments only—Raw values).

FIG. 6i depicts the graphs showing secreted Collagen 1a1 levels in PCLS culture supernatants (Tranilast (31.25 μM) with nintedanib Treatments only—% change from baseline).

FIG. 6j depicts the graphs showing secreted Collagen 1a1 levels in PCLS culture supernatants (Tranilast (125 μM) with nintedanib Treatments only—% change from baseline).

FIG. 7a shows a comparison of secreted Col1a1 levels in PCLS culture supernatants after 4-day dosing of tranilast (TL) and Pirfenidone (Pir) alone and in combination.

FIG. 7b shows a comparison of secreted Col 1a1 levels in PCLS culture supernatants after 4-day dosing of tranilast (TL) and nintedanib (Nin) alone and in combination.

FIG. 8 shows Col1A1 secretions as a percent of baseline at T=144 hours (6 days) as a mean of 6 slices.

### DETAILED DESCRIPTION

The invention relates to the treatment of idiopathic pulmonary fibrosis (IPF) using a therapeutically effective amount of a combination of tranilast and pirfenidone, or a therapeutically effective amount of a combination of tranilast and nintedanib. Each active pharmaceutical ingredient (API) used in a combination of the invention, pirfenidone, nintedanib, or tranilast, may be used as the compound itself (e.g., a free base or free acid), as a salt form, or in any other known form. For example, nintedanib may be used as nintedanib esylate. Tranilast itself may be used or may be in the form of a tranilast salt or a tranilast cocrystal such as those discussed below. Regarding the dose amounts or molar ratios discussed below, the amounts or molar ratios refer to the amount of the API's, pirfenidone, nintedanib, and tranilast, whether the API is present itself, as a salt, or a cocrystal.

Tranilast, (2-[[3-(3,4-dimethoxyphenyl)-1-oxo-2-propenyl]amino]benzoic acid), shown below, is a therapeutic agent that exhibits an anti-allergic effect. Tranilast has been used as an anti-allergic treatment, for several years in Japan and South Korea, for conditions such as allergic conjunctivitis, bronchial asthma, allergic rhinitis, and atopic dermatitis.

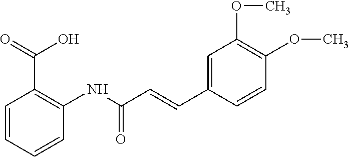 [SMILES: COc1ccc(/C=C/C(=O)Nc2ccccc2C(=O)O)cc1OC]

Additionally, tranilast has been demonstrated to possess anti-fibrotic, anti-inflammatory, anti-tumor, neurogenesis enhancing and angiogenesis inhibitory properties and as such may be useful for the treatment of diseases associated with such properties. Tranilast and pharmaceutically acceptable salts of tranilast are described in EP 1 946 753 A1 and in US 2011/0136835 A1 (which also mentions a tranilast cocrystal) both of which are incorporated herein by reference.

WO 2013/144916 A1, incorporated herein by reference, reports several tranilast complexes and several tranilast cocrystals as well as pharmaceutical compositions containing them. In particular, WO 2013/144916 A1 discloses a 1:1 tranilast nicotinamide complex, a 1:1 tranilast nicotinamide cocrystal, a 1:1 tranilast saccharin complex, a 1:1 tranilast saccharin cocrystal, a 1:1 tranilast gentisic acid complex, a 1:1 tranilast gentisic acid cocrystal, a 1:1 tranilast salicylic acid complex, a 1:1 tranilast salicylic acid cocrystal, a 1:1 tranilast urea complex, a 1:1 tranilast urea cocrystal, a 1:1 tranilast 4-aminobenzoic acid complex, a 1:1 tranilast 4-aminobenzoic acid cocrystal, a 1:1 tranilast 2,4-dihydroxybenzoic acid complex and a 1:1 tranilast 2,4-dihydroxybenzoic acid cocrystal. The various pharmaceutical compositions containing these tranilast complexes and cocrystals disclosed in WO 2013/144916 A1 include inhalable compositions among others.

WO 2020/035546 A1, incorporated herein by reference, reports several tranilast salts as well crystalline forms of those salts and pharmaceutical compositions containing them. In particular, WO 2020/035546 A1 discloses a crystalline 1:1 tranilast n-methylglucamine form I salt; a crystalline 1:1 tranilast n-methylglucamine form II salt; a crystalline 1:1 tranilast I-lysine salt; a crystalline 1:1 tranilast diethylamine salt; a crystalline 1:1 tranilast n-ethylglucamine salt; a crystalline 1:1 tranilast potassium monohydrate salt; a crystalline 1:1 tranilast diethanolamine salt; and a crystalline 1:1 tranilast ethanolamine salt. The various pharmaceutical compositions containing these tranilast complexes and cocrystals disclosed in WO 2020/035546 A1 include inhalable compositions among others. The use of a 1:1 tranilast I-lysine salt, particularly a crystalline 1:1 tranilast I-lysine salt, is a preferred form of tranilast for use in the various embodiments of this invention.

### Treatment of Idiopathic Pulmonary Fibrosis (IPF)

The invention relates to a method of treating idiopathic pulmonary fibrosis (IPF) by administering to a patient in need thereof a combined amount of tranilast and pirfenidone therapeutically effective to treat idiopathic pulmonary fibrosis (IPF). The invention relates to the therapeutic use of a combined amount of tranilast and pirfenidone therapeutically effective to treat idiopathic pulmonary fibrosis (IPF). In a method or use of the invention tranilast may be administered as an adjunctive therapy to to the standard of care for pirfenidone treatment of IPF. A method of treatment or a use of the invention may contain a non-therapeutic amount of pirfenidone or tranilast in comparison to either API when administered to a patient individually to treat IPF. In other embodiments, a combination of tranilast and nintedanib used may also be synergistically effective to treat IPF. In such a method of treatment or a use of the invention the tranilast and pirfenidone are administered in a combined amount synergistically effective to treat IPF.

The invention relates to a method of treating idiopathic pulmonary fibrosis (IPF) by administering to a patient in need thereof a combined amount of tranilast and nintedanib therapeutically effective to treat idiopathic pulmonary fibrosis (IPF). The invention relates to the therapeutic use of a combined amount of tranilast and nintedanib therapeutically effective to treat idiopathic pulmonary fibrosis (IPF). In a method or use of the invention tranilast may be administered as an adjunctive therapy to the standard of care for nintedanib treatment of IPF. A method of treatment or a use of the invention may contain a non-therapeutic amount of nintedanib or tranilast in comparison to either API when administered to a patient individually to treat IPF. In other embodiments, a combination of tranilast and nintedanib used may also be synergistically effective to treat IPF. In such a method of treatment or a use of the invention the tranilast and nintedanib are administered in a combined amount synergistically effective to treat IPF.

The invention also relates to a pharmaceutical package containing such different pharmaceutical compositions of tranilast and pirfenidone or of tranilast and nintedanib (e.g, an inhalable formulation of tranilast and a pill or capsule of pirfenidone or nintedanib) which when administered is therapeutically effective to treat IPF.

The term “treatment” or “treating” means any treatment of a disease, disorder or condition in a mammal, including: preventing or protecting against the disease, disorder or condition, that is, causing the clinical symptoms not to develop; inhibiting the disease, disorder or condition, that is, arresting or suppressing the development of clinical symptoms; and/or relieving the disease, disorder or condition (including the relief of discomfort associated with the condition or disorder), that is, causing the regression of clinical symptoms. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” the disease, disorder, or condition. The term “protection” is meant to include “prophylaxis.”

The actual amount of a therapeutic or, as in this invention, a combination of therapeutics, required for treatment of any particular disease, disorder or condition for any particular patient may depend upon a variety of factors including, for example, the particular disease, disorder or condition being treated; the disease state being treated and its severity; the specific pharmaceutical composition employed; the age, body weight, general health, sex and diet of the patient; the mode of administration; the time of administration; the route of administration; and the rate of excretion of an API; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman, and L. Limbird, eds., McGraw-Hill Press, 155-173, 2001, which is incorporated herein by reference. As discussed above, tranilast may be administered as itself, as a tranilast salt, or as a tranilast cocrystal. Tranilast is marketed in Japan and South Korea by Kissei Pharmaceutical Co. Ltd under the Rizaben® brand name. Tranilast is prescribed orally to treat bronchial asthma, allergic rhinitis, atopic dermatitis, keloid, or hypertrophic scar. The typical dosage in adults for these conditions is currently one 100 mg tablet three times per day. However, it was shown that a dose of at least 600 mg per day was required to treat a proliferative disease such as restenosis (H. Tamai, Am Heart J. 1999; 138(5): 968-75) and a Phase 11 clinical study of tranilast for the prevention of restenosis included doses as high as 900 mg per day (D Holmes, Circulation. 2002; 106(10): 1243-1250).

To treat IPF, pirfenidone, as the ESBRIET therapeutic, has a recommended oral daily dosage of 2403 mg/day given in three doses of 801 mg per day. Nintedanib, as the OFEV therapeutic used to treat IPF, has a recommended adult oral daily dosage of 300 mg/day taken twice daily in 150 mg doses. Though not used to treat IPF, tranilast has a recommended oral daily dosage of 300 mg/day, administered three times per day in 100 mg doses to treat allergic disorders. In a method of treatment or therapeutic use, the amount of pirfenidone and nintedanib in a combination of the invention administered daily is less than or equal to the amount of that API if individually administered to treat IPF. Tranilast is administered in an amount to achieve the beneficial effect of the combination, generally in an amount ranging from about 5 mg to 600 mg. The amount of tranilast administered may be adjusted is the dose of pirfenidone or nintedanib is adjusted over the course of treatment.

According to the invention, tranilast may be administered as an adjunctive therapy to pirfenidone treatment of IPF or as an adjunctive therapy to nintedanib treatment of IPF. The combination of tranilast and pirfenidone or the combination of tranilast and nintedanib may be administered in the same or different pharmaceutical compositions and/or by the same or different routes of administration. For example, tranilast may be administered as an inhaled pharmaceutical composition and pirfenidone or nintedanib orally administered or may be orally administered in the same or separate pills or capsules.

An adjunctive therapeutic method of the invention to treat idopathic pulmonary fibrosis (IPF) administers to a patient in need thereof an oral dosage of pirefenidone effective to treatment idiopathic pulmonary fibrosis (IPF) and an inhaled dosage of tranilast therapeutically effective to treat idiopathtic pulmonary fibrosis (IPF) in combination with the adminstered pirfenidone. Another adjunctive therapeutic method of the invention to treat idopathic pulmonary fibrosis (IPF) administers to a patient in need thereof an oral dosage of nintedanib effective to treatment idiopathic pulmonary fibrosis (IPF) and an inhaled dosage of tranilast therapeutically effective to treat idiopathtic pulmonary fibrosis (IPF) in combination with the adminstered nintedanib. In an adjunctive therapeutic method of the invention the tranilast administered is a tranilast cocrystal or a tranilast salt, preferably a 1:1 tranilast I-lysine salt or a crystalline 1:1 tranilast I-lysine salt, administered as a liquid inhalation formulation. The nintedanib administered is nintedanib esylate. Exemplary doses of tranilast, pirfenidone and nintedanib used in an and junctive therapy are listed as daily doses in Table 1. For example, when pirfenidone is administered in a daily amount of 2403 mg, the amount of tranilast may be 50 mg-600 mg, 200 mg-500 mg, 300 mg, or 100 mg. The daily dose may be a single dose or divided among multiple sub-doses throughout the day, e.g. two sub-doses, three sub-doses, four sub-doses, etc.

TABLE 1
Exemplary Daily Adjunctive Therapy Doses

| API Daily Doses | Tranilast | Tranilast | Tranilast | Tranilast |
|---|---|---|---|---|
| Pirfenidone | | | | |
| 2403 mg | 50 mg to 600 mg | 200 mg to 500 mg | 300 mg | 100 mg |
| 1200 mg to 2410 | 50 mg to 600 mg | 200 mg to 500 mg | 50 mg to 300 mg | 100 mg or 300 mg |
| mg | | | | |
| 750 mg to 1205 | 50 mg to 600 mg | 200 mg to 500 mg | 50 mg to 300 mg | 100 mg or 300 mg |
| mg | | | | |
| 801 mg | 50 mg to 600 mg | 200 mg to 500 mg | 50 mg to 300 mg | 100 mg or 300 mg |
| Nintedanib | | | | |
| 300 mg | 50 mg to 600 mg | 200 mg to 500 mg | 300 mg | 100 mg |
| 100 mg to 300 mg | 50 mg to 600 mg | 200 mg to 500 mg | 50 mg to 300 mg | 100 mg or 300 mg |
| 150 mg | 50 mg to 600 mg | 200 mg to 500 mg | 50 mg to 300 mg | 100 mg or 300 mg |

In a method of treatment or therapeutic use of either a combination of tranilast and pirfenidone or a combination of tranilast and nintedanib, the amount of each API, tranilast, pirfenidone, and nintedanib, administered is less than the amount of that API if individually administered to treat IPF. In other words, the amount of each API in a combination of the invention may be a sub-optimal amount for the therapeutic treatment of IPF by the API alone. It is the combined amount of tranilast and pirfenidone or of nintedanib and tranilast that is therapeutically effective to treat IPF. The efficacy of the combined API's, pirfenidone and tranilast or nintedanib and tranilast, is greater than the efficacy when the amount of each API in a combination is administered individually. As shown in the examples below, the combination of tranilast and pirfenidone and the combination of tranilast and nintedanib achieve increased therapeutic efficacy over the individual API's. The combinations of the invention even achieve a synergistic therapeutic efficacy in the treatment of IPF.

When administering combinations of sub-opimal therapeutic amounts or synergistic amounts, the combination of tranilast and pirfenidone or the combination of tranilast and nintedanib may be administered in the same or different pharmaceutical compositions and/or by the same or different routes of administration. For example, tranilast may be administered as an inhaled pharmaceutical composition and pirfenidone or nintedanib orally administered or may be orally administered in the same or separate pills or capsules. A therapeutic method of the invention to treat idopathic pulmonary fibrosis (IPF) administers to a patient in need thereof an oral dosage of pirefenidone effective to treatment idiopathic pulmonary fibrosis (IPF) and an inhaled dosage of tranilast therapeutically effective to treat idiopathtic pulmonary fibrosis (IPF) in combination with the adminstered pirfenidone. Another therapeutic method of the invention to treat idopathic pulmonary fibrosis (IPF) administers to a patient in need thereof an oral dosage of nintedanib effective to treatment idiopathic pulmonary fibrosis (IPF) and an inhaled dosage of tranilast therapeutically effective to treat idiopathtic pulmonary fibrosis (IPF) in combination with the adminstered nintedanib. In a therapeutic method of the invention the tranilast administered is a tranilast cocrystal or a tranilast salt, preferably a 1:1 tranilast I-lysine salt or a crystalline 1:1 tranilast I-lysine salt, administered as a liquid inhalation formulation. The nintedanib administered is nintedanib esylate.

Table 2 shows exemplary molar ratios of tranilast to pirfenidone and of tranilast to nintedanib in the combinations of the invention. Each molar ratio represents a separate embodiment of the invention and encompasses synergistic combinations. For example the molar ratio of tranilast to pirfenidone may range from 1:500 to 500:1, 1:200 to 200:1, 1:100 to 100:1, 1:50 to 50:1, 1:25 to 25:1, 1:10 to 10:1, 5:1 to 1:5, 2:1 to 1:2, or 1:1. Table 3 shows exemplary daily doses of the combination of pirfenidone and tranilast and of the combination of tranilast and nintedanib. Each combination in the table represents a separate embodiment of the invention and encompasses synergistic combinations. For example, when nintedanib is administered in a daily amount of 10 mg to 200 mg mg, the amount of tranilast may be 5 mg-00 mg, 5 mg-250 mg, 10 mg-300 mg, 50 mg-200 mg, 10 mg-100 mg, 5 mg-50 mg, or 100 mg-250 mg. The daily dose may be a single dose or divided among multiple sub-doses throughout the day, e.g. two sub-doses, three sub-doses, four sub-doses, etc.

TABLE 2
Exemplary Molar Ratios

| API | Molar ratio tranilast:API | | | | | | | | |
|---|---|---|---|---|---|---|---|---|---|
| Pirfenidone | 1:500 to | 1:200 to | 1:100 to | 1:50 to | 1:25 to | 1:10 to | 5:1 to | 2:1 to | 1:1 |
| | 500:1 | 200:1 | 100:1 | 50:1 | 25:1 | 10:1 | 1:5 | 1:2 | |
| Nintedanib | 1:500 to | 1:200 to | 1:100 to | 1:50 to | 1:25 to | 1:10 to | 5:1 to | 2:1 to | 1:1 |
| | 500:1 | 200:1 | 100:1 | 50:1 | 25:1 | 10:1 | 1:5 | 1:2 | |

TABLE 3
Exemplary Daily Dose Combinations

| API Daily | | | | | | | |
|---|---|---|---|---|---|---|---|
| Doses | Tranilast | Tranilast | Tranilast | Tranilast | Tranilast | Tranilast | Tranilast |
| Pirfenidone | | | | | | | |
| 50 mg to | 5 mg to | 5 mg to | 10 mg to | 50 mg to | 10 mg to | 5 mg to | 100 mg to |
| 2300 mg | 500 mg | 250 mg | 300 mg | 200 mg | 100 mg | 50 mg | 250 mg |
| 100 mg to | 5 mg to | 5 mg to | 10 mg to | 50 mg to | 10 mg to | 5 mg to | 100 mg to |
| 1500 mg | 500 mg | 250 mg | 300 mg | 200 mg | 100 mg | 50 mg | 250 mg |
| 500 mg to | 5 mg to | 5 mg to | 10 mg to | 50 mg to | 10 mg to | 5 mg to | 100 mg to |
| 1000 mg | 600 mg | 250 mg | 300 mg | 200 mg | 100 mg | 50 mg | 250 mg |
| 300 mg to | 5 mg to | 5 mg to | 10 mg to | 50 mg to | 10 mg to | 5 mg to | 100 mg to |
| 800 mg | 600 mg | 250 mg | 300 mg | 200 mg | 100 mg | 50 mg | 250 mg |
| 750 mg to | 5 mg to | 5 mg to | 10 mg to | 50 mg to | 10 mg to | 5 mg to | 100 mg to |
| 1250 mg | 600 mg | 250 mg | 300 mg | 200 mg | 100 mg | 50 mg | 250 mg |
| 100 mg to | 5 mg to | 5 mg to | 10 mg to | 50 mg to | 10 mg to | 5 mg to | 100 mg to |
| 600 mg | 600 mg | 250 mg | 300 mg | 200 mg | 100 mg | 50 mg | 250 mg |
| Nintedanib | | | | | | | |
| 5 mg to 275 | 5 mg to | 5 mg to | 10 mg to | 50 mg to | 10 mg to | 5 mg to | 100 mg to |
| mg | 600 mg | 250 mg | 300 mg | 200 mg | 100 mg | 50 mg | 250 mg |
| 10 mg to 200 | 5 mg to | 5 mg to | 10 mg to | 50 mg to | 10 mg to | 5 mg to | 100 mg to |
| mg | 600 mg | 250 mg | 300 mg | 200 mg | 100 mg | 50 mg | 250 mg |
| 25 mg to 150 | 5 mg to | 5 mg to | 10 mg to | 50 mg to | 10 mg to | 5 mg to | 100 mg to |
| mg | 600 mg | 250 mg | 300 mg | 200 mg | 100 mg | 50 mg | 250 mg |
| 50 mg to 175 | 5 mg to | 5 mg to | 10 mg to | 50 mg to | 10 mg to | 5 mg to | 100 mg to |
| mg | 600 mg | 250 mg | 300 mg | 200 mg | 100 mg | 50 mg | 250 mg |
| 100 mg to | 5 mg to | 5 mg to | 10 mg to | 50 mg to | 10 mg to | 5 mg to | 100 mg to |
| 225 mg | 600 mg | 250 mg | 300 mg | 200 mg | 100 mg | 50 mg | 250 mg |
| 75 mg to 125 | 5 mg to | 5 mg to | 10 mg to | 50 mg to | 10 mg to | 5 mg to | 100 mg to |
| mg | 600 mg | 250 mg | 300 mg | 200 mg | 100 mg | 50 mg | 250 mg |

In a method of treatment or therapeutic use according to the invention a combination of tranilast and pirfenidone, or a combination of tranilast and nintedanib, may be administered as a pharmaceutical composition including but not limited to a tablet, a capsule, an oral solution, an inhalable formulation (e.g. an inhalable powder or an inhalable solution), or an injectable composition. A combination of tranilast and pirfenidone, or a combination of tranilast and nintedanib, may be administered in a single pharmaceutical composition or in separate pharmaceutical compositions. The pharmaceutical composition may be an oral composition or, if administered separately, using pharmaceutical compositions known for the particular API. For example, tranilast may be administered as an inhalable powder or an inhalable solution while the pirfenidone or the nintedanib is administered orally as a tablet or capsule. Or, tranilast and pirfenidone or tranilast and nintedanib may each be administered in individual tablets or capsules or as individual inhalable formulations.

There are various methods of administering a combination of tranilast and pirfenidone, or a combination of tranilast and nintedanib, according to the invention topically to the lung. One such means could involve a dry powder inhalable formulation of respirable particles comprising, consisting essentially of, or consisting of an API such as tranilast, a combination of tranilast and pirfenidone, or a combination of tranilast and nintedanib according to the invention which the patient being treated inhales for nasal treatment. It is common for a dry powder formulation to include carrier particles, to which the API can adhere to. The carrier particles may be of any acceptable pharmacologically inert material or combination of materials. For example, the carrier particles may be composed of one or more materials selected from sugar alcohols; polyols, for example sorbitol, mannitol or xylitol, and crystalline sugars, including monosaccharides and disaccharides; inorganic salts such as sodium chloride and calcium carbonate; organic salts such as sodium lactate; and other organic compounds such as urea, polysaccharides, for example cyclodextrins and dextrins. The carrier particles may be a crystalline sugar, for example, a monosaccharide such as glucose or arabinose, or a disaccharide such as maltose, saccharose, dextrose or lactose. The crystalline tranilast salt would be dispersed into the respiratory tract, and subsequently contact the lower lung in a pharmaceutically effective amount. Another such means could involve a solution inhalable formulation comprising, consisting essentially of, or consisting of an API such as tranilast, a combination of tranilast and pirfenidone, or a combination of tranilast and nintedanib, according to the invention which the patient being treated inhales for nasal treatment thereby delivering the API to the respiratory tract and subsequently to the lower lungs.

### Pharmaceutical Compositions

The invention relates to a pharmaceutical composition comprising, consisting essentially of, or consisting of a therapeutically effective amount of tranilast and pirfenidone in a combined amount therapeutically effective to treat idiopathic pulmonary fibrosis (IPF). In a pharmaceutical composition of the invention the tranilast and pirfenidone may be present in a combined amount synergistically effective to treat IPF. A pharmaceutical composition of the invention may contain a non-therapeutic amount of pirfenidone or tranilast in comparison to either API when administered to a patient individually to treat IPF.

The invention relates to a pharmaceutical composition comprising, consisting essentially of, or consisting of a therapeutically effective amount of tranilast and nintedanib in a combined amount therapeutically effective to treat idiopathic pulmonary fibrosis (IPF). In a pharmaceutical composition of the invention the tranilast and nintedanib may be present in a combined amount synergistically effective to treat IPF. A pharmaceutical composition of the invention may contain a non-therapeutic amount of nintedanib or tranilast in comparison to either API when administered to a patient individually to treat IPF.

The invention also relates to pharmaceutical compositions comprising, consisting essentially of, or consisting of, as the API, a therapeutically effective amount of a combination of tranilast and pirfenidone, or a therapeutically effective amount of a combination of tranilast and nintedanib, and a pharmaceutically acceptable carrier (also known as a pharmaceutically acceptable excipient). When a pharmaceutical composition contains a combination of tranilast and pirfenidone or a combination of tranilast and nintedanib that combination may be a synergistically therapeutically effective to treat idiopathic pulmonary fibrosis (IPF).

A pharmaceutical composition of the invention may be in any pharmaceutical form which contains a therapeutically effective amount of a combination of tranilast and pirfenidone, or a therapeutically effective amount of a combination of tranilast and nintedanib, particularly the synergistic combinations, according to the invention. The pharmaceutical composition may be, for example but not limited to, a tablet, a capsule, an oral solution, an inhalable formulation (an inhalable powder or an inhalable solution), or an injectable composition. The pharmaceutical compositions generally contain, for example, about 0.1% to about 99.9% by weight of a combination of tranilast and pirfenidone, or of a combination of tranilast and nintedanib, for example, about 0.5% to about 99% by weight of such a combination of tranilast and pirfenidone or of tranilast and nintedanib, and, for example, 99.5% to 0.5% by weight of at least one suitable pharmaceutical excipient or solvent. In one embodiment, the composition may be between about 5% and about 75% by weight of a combination of tranilast and pirfenidone or of tranilast and nintedanib of the invention with the rest being at least one suitable pharmaceutical excipient, solvent or at least one other adjuvant, as discussed below.

The pharmaceutical compositions of the invention may be prepared by methods known in the pharmaceutical formulation art, for example, see Remington's Pharmaceutical Sciences, 18th Ed., (Mack Publishing Company, Easton, Pa., 1990), which is incorporated herein by reference. In a solid dosage form, a therapeutically effective amount of a combination of tranilast and pirfenidone, or a therapeutically effective amount of a combination of tranilast and nintedanib, particularly the synergistic combinations, of the invention may be admixed with at least one pharmaceutically acceptable excipient such as, for example, sodium citrate or dicalcium phosphate or (a) fillers or extenders, such as, for example, starches, lactose, sucrose, glucose, mannitol, and silicic acid, (b) binders, such as, for example, cellulose derivatives, starch, alginates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents.

Pharmaceutically acceptable adjuvants known in the pharmaceutical formulation art may also be used in the pharmaceutical compositions of the invention. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms are one embodiment of the pharmaceutical composition of the invention. Dosage forms for oral administration, which includes capsules, tablets, pills, powders, granules, and suspensions may be used. Dosage forms for pulmonary administration, which includes metered dose inhaler, dry powder inhaler or aerosol formulations may be used. In such solid dosage forms, the active pharmaceutical ingredients (tranilast, pirfenidone, and/or nintedanib) may be mixed with at least one inert, pharmaceutically acceptable excipient (also known as a pharmaceutically acceptable carrier). Solid dosage forms may be prepared with coatings and shells, such as enteric coatings and others, as is known in the pharmaceutical art. They may contain pacifying agents and can also be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active pharmaceutical ingredients (tranilast, pirfenidone, and/or nintedanib), may contain suspending agents, such as, for example, ethoxylated isostearyl alcohols, polyoxyethylene sorbitol and sorbitan esters, microcrystalline cellulose, aluminum metal hydroxide, bentonite, agar-agar and tragacanth, or mixtures of these substances, and the like. Liquid dosage forms may be aqueous, may contain a pharmaceutically acceptable solvent as well as traditional liquid dosage form excipients known in the art which include, but are not limited to, buffering agents, flavorants, sweetening agents, preservatives, and stabilizing agents.

The active pharmaceutical ingredients (tranilast, pirfenidone, and/or nintedanib) may also be formulated as liquid or injectable pharmaceutical composition. Administration in pure form, as a salt, as a cocrystal, or in an appropriate pharmaceutical composition may be carried out via any of the accepted modes of administration or agents for serving similar utilities. Thus, administration may be, for example, orally, buccally, nasally, pulmonary, parenterally (intravenous, intramuscular, or subcutaneous), topically, transdermally, intravaginally, intravesically, intrasystemically, ophthalmically, or rectally, in the form of solid, semi-solid, lyophilized powder, or liquid dosage forms, such as, for example, tablets, suppositories, pills, soft elastic and hard gelatin capsules, powders, solutions, suspensions, or aerosols, or the like, such as, for example, in unit dosage forms suitable for simple administration of precise dosages. One route of administration may be oral administration, using a convenient daily dosage regimen that can be adjusted according to the degree of severity of the condition to be treated.

For conditions such as fibrosis, where the patient may be unable to swallow solid oral dosage forms, a pharmaceutical composition of the active pharmaceutical ingredients (tranilast, pirfenidone, and/or nintedanib) may be in a liquid form, such as for example an injectable or a liquid oral formulation that can be administered by a healthcare specialist.

The invention also relates to a method of preparing a liquid pharmaceutical composition comprising, consisting essentially of, or consisting of the step of dissolving an active pharmaceutical ingredient or a combination according to the invention in a pharmaceutically acceptable solvent and to liquid pharmaceutical compositions prepared according to that method. Physiologically acceptable aqueous vehicles may be used as the solvent to prepare a liquid formulation of the invention. Water or pharmaceutically acceptable isotonic aqueous solution is preferred. As discussed above, liquid pharmaceutical compositions of the invention may be administered orally, parenterally (including by inhalation), and intravenously.

### EXAMPLES

### Example 1—Human IPF-PCLS Model for Inflammation and Fibrosis

Combinations of a therapeutic agent, pirfenidone or nintedanib, were tested in the presence or absence of Tranilast for the ability to attenuate inflammation and fibrosis in a human IPF-PCLS model.

Precision Cut lung Slices (PCLS) from human lung tissue are physiologically and structurally representative of the tissue architecture. Testing potential therapeutic targets and interrogating mechanisms underpinning disease pathophysiology in human PCLS allows assessment of the combined therapeutic agents' effectiveness and relevance to clinical situations, overcoming many of the limitations of currently widely employed in vivo rodent models and in vitro 2D cell culture methodologies.

### 1.1 Materials and Methods

PCLS preparation and culture: PCLS were prepared from a single explanted human IPF lung and rested for 48 hrs to allow the post-slicing stress period to elapse before experiments began. PCLS were subsequently incubated with vehicle (0.1% DMSO), tranilast as a 1:1 l-lysine tranilast salt at 2 escalating concentrations (31.25 μM, 125 μM), pirfenidone at 3 escalating concentrations (0.25 mM, 1 mM, 2.5 mM) or nintedanib at 3 escalating concentrations (0.25 μM, 1 μM, 2.5 μM) in the presence or absence of Tranilast at 2 escalating concentrations (31.25 μM, 125 μM) for a further 96 hrs. PCLS culture media and all compounds were refreshed at 24 hrs intervals. All PCLS were harvested at 144 hrs.

Experimental groups: Twenty-one (21) different groups with n=6 human PCLS (total n=126 per lung) were investigated as shown in Table 4 below. PCLS were prepared from n=1 individual human IPF lungs.

TABLE 4
Experimental Groups

| | | | | Co- | | | Experimental | | |
|---|---|---|---|---|---|---|---|---|---|
| Group | Treatment | Concentration | | treatment | Concentration | | Period | Harvest | PCLS |
| 1 | — | — | | — | — | | 48-144 hrs | 144 hrs | n = 6 |
| 2 | — | — | | Tranilast | 31.25 | μM | 48-144 hrs | 144 hrs | n = 6 |
| 3 | — | — | | Tranilast | 125 | μM | 48-144 hrs | 144 hrs | n = 6 |
| 4 | pirfenidone | 0.25 | mM | — | — | | 48-144 hrs | 144 hrs | n = 6 |
| 5 | pirfenidone | 1 | mM | — | — | | 48-144 hrs | 144 hrs | n = 6 |
| 6 | pirfenidone | 2.5 | mM | — | — | | 48-144 hrs | 144 hrs | n = 6 |
| 7 | pirfenidone | 0.25 | mM | Tranilast | 31.25 | μM | 48-144 hrs | 144 hrs | n = 6 |
| 8 | pirfenidone | 1 | mM | Tranilast | 31.25 | μM | 48-144 hrs | 144 hrs | n = 6 |
| 9 | pirfenidone | 2.5 | mM | Tranilast | 31.25 | μM | 48-144 hrs | 144 hrs | n = 6 |
| 10 | pirfenidone | 0.25 | mM | Tranilast | 125 | μM | 48-144 hrs | 144 hrs | n = 6 |
| 11 | pirfenidone | 1 | mM | Tranilast | 125 | μM | 48-144 hrs | 144 hrs | n = 6 |
| 12 | pirfenidone | 2.5 | mM | Tranilast | 125 | μM | 48-144 hrs | 144 hrs | n = 6 |
| 13 | nintedanib | 0.25 | μM | — | — | | 48-144 hrs | 144 hrs | n = 6 |
| 14 | nintedanib | 1 | μM | — | — | | 48-144 hrs | 144 hrs | n = 6 |
| 15 | nintedanib | 2.5 | μM | — | — | | 48-144 hrs | 144 hrs | n = 6 |
| 16 | nintedanib | 0.25 | μM | Tranilast | 31.25 | μM | 48-144 hrs | 144 hrs | n = 6 |
| 17 | nintedanib | 1 | μM | Tranilast | 31.25 | μM | 48-144 hrs | 144 hrs | n = 6 |
| 18 | nintedanib | 2.5 | μM | Tranilast | 31.25 | μM | 48-144 hrs | 144 hrs | n = 6 |
| 19 | nintedanib | 0.25 | μM | Tranilast | 125 | μM | 48-144 hrs | 144 hrs | n = 6 |
| 20 | nintedanib | 1 | μM | Tranilast | 125 | μM | 48-144 hrs | 144 hrs | n = 6 |
| 21 | nintedanib | 2.5 | μM | Tranilast | 125 | μM | 48-144 hrs | 144 hrs | n = 6 |

| | |
|---|---|
| Total | n = 126 |

PCLS Treatments and Media Collections: PCLS were incubated for a 48 hr rest period. Post-rest, PCLS were incubated for a further 96 hrs in the presence or absence of inhibitors as outlined above. PCLS culture media, including all compounds, were refreshed and harvested at 24 hrs intervals from 48 hrs. All PCLS were harvested at 144 hrs.

### 1.2 Results

PCLS harvest: Cell culture supernatant (n=6 per group) were collected every 24 hrs and snap frozen for quantification of inflammatory cytokines and soluble ECM proteins. At harvest, PCLS (n=6 per group) and cell culture media (n=6 per group) were snap frozen for quantification of inflammatory cytokines and soluble ECM proteins.

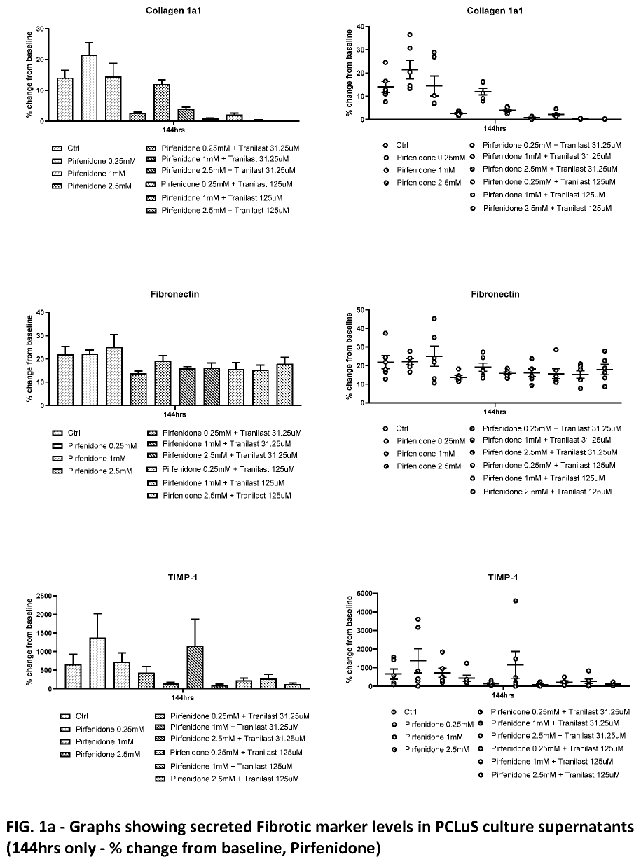

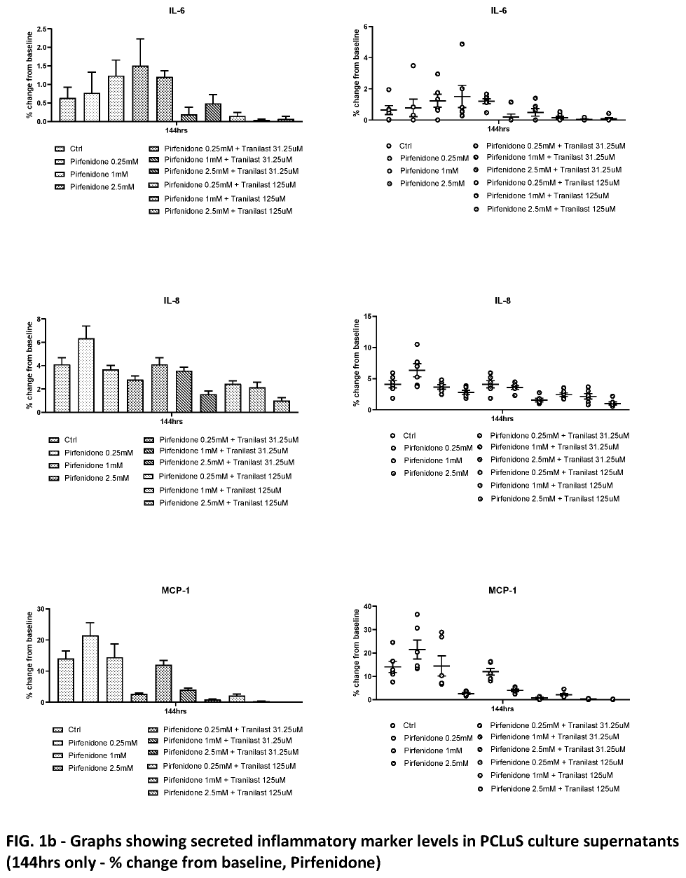

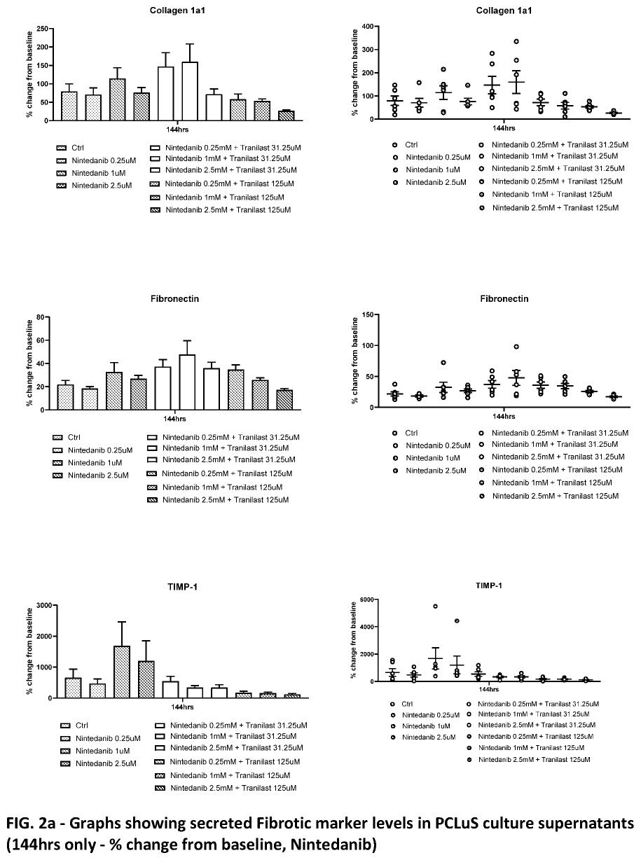

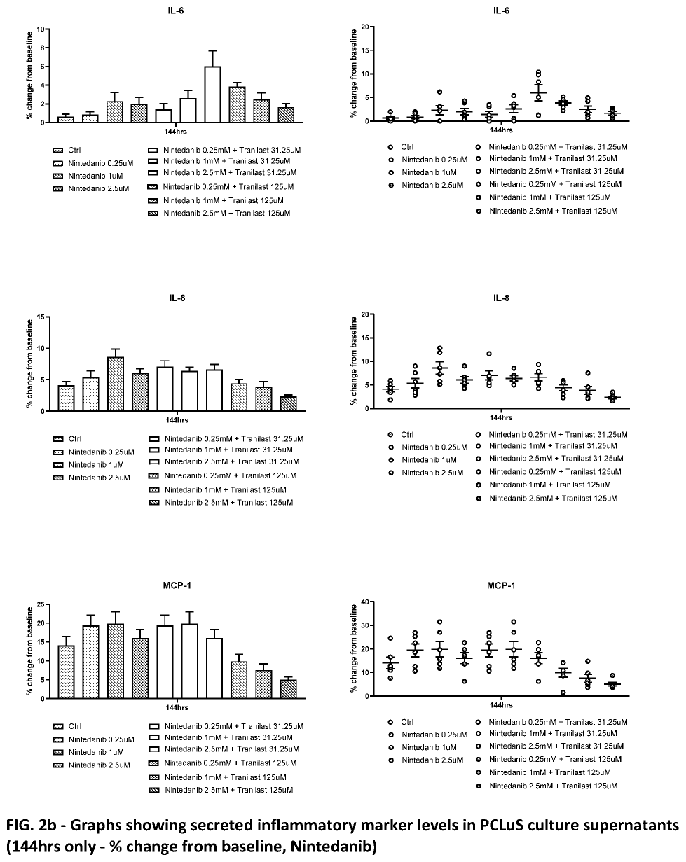

Soluble outputs analysis: Anti-inflammatory potential was assessed by determining the levels of the inflammation markers MCP-1, IL-6, GMCSF, IL-8, and IL-1β in the tissue culture supernatants experiment (n=213 samples (n=630 samples; n=126 at 48 hrs, 72 hrs, 96 hrs, 120 hrs and 144 hrs) were quantified using a MSD multiplex ELISA (MesoScaleDiscovery). Anti-fibrotic potential was assessed by determining the levels of the fibrosis markers Collagen-1a1, TIMP1, and Fibronectin in the tissue culture supernatants (n=630 samples as above) were quantified using a single-plex ELISA (R&D). Summary data for all inflammatory markers and fibrotic markers in PCLS treated with pirfenidone (FIGS. 1a and 1b) or nintedanib (FIGS. 2a and 2b) in the presence or absence of Tranilast at 144 hrs only. Graphs are represented by n=6 tissue culture media per condition, per time point. Data is presented as mean±SEM with individual values per tissue culture media.

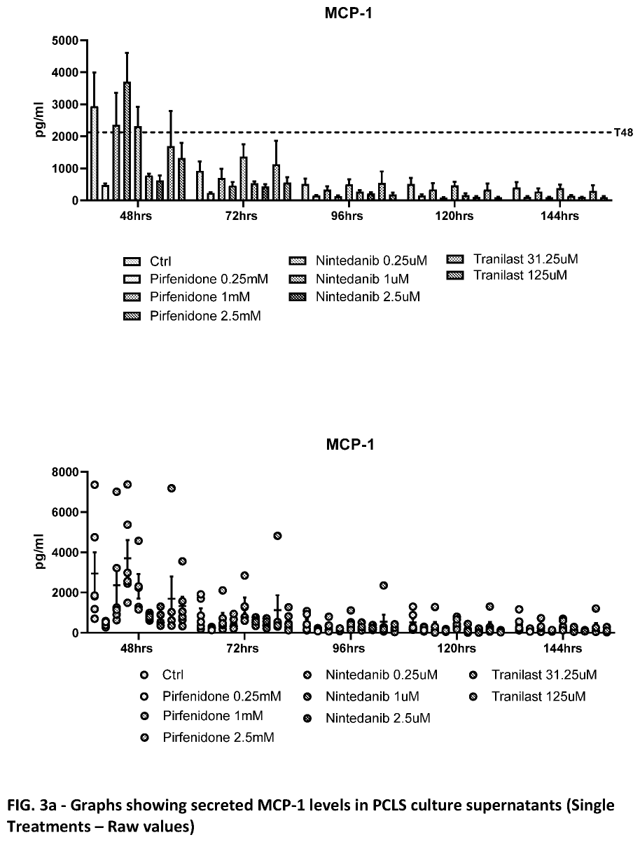

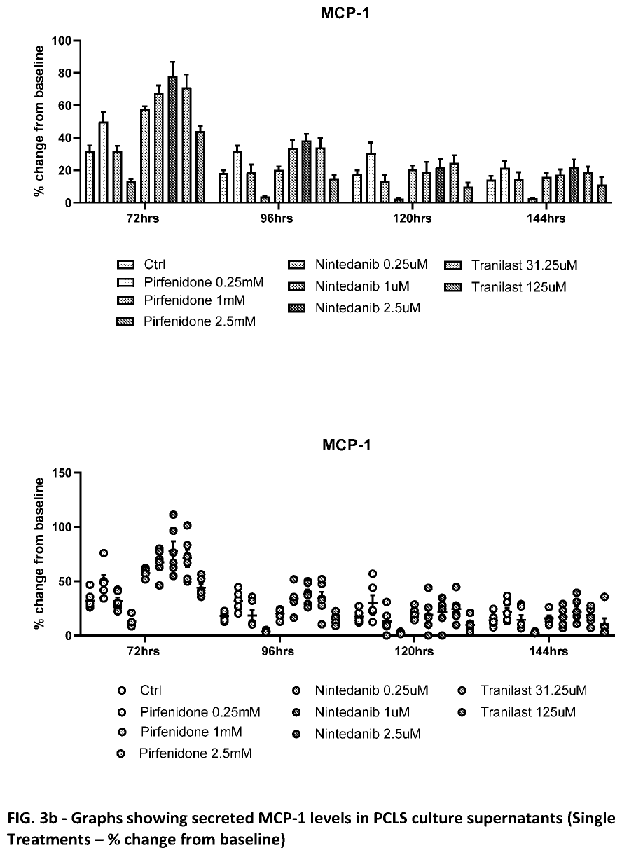

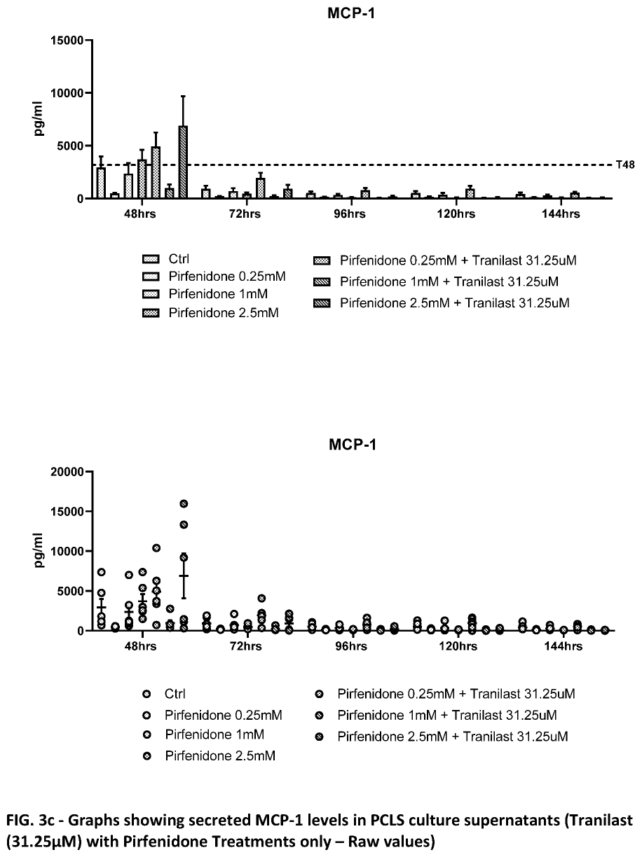

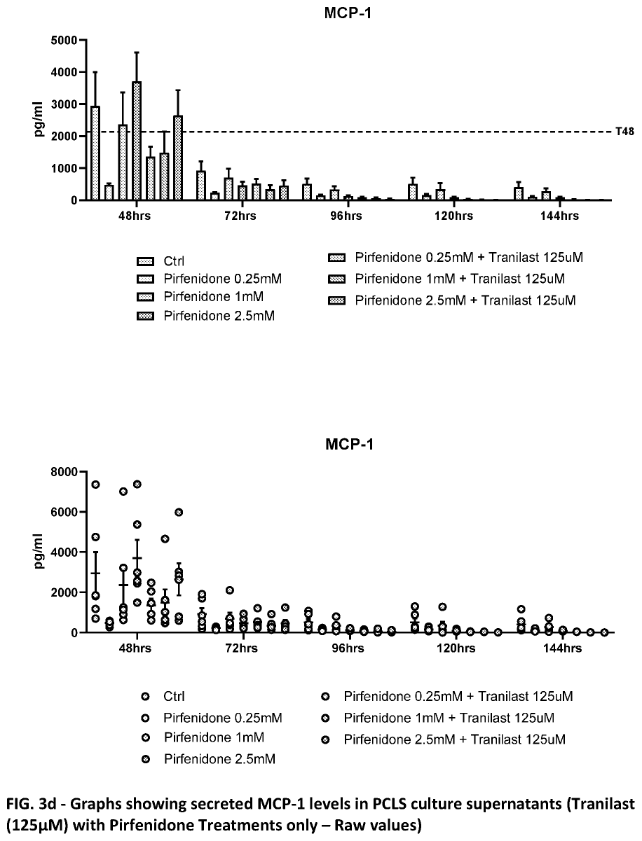

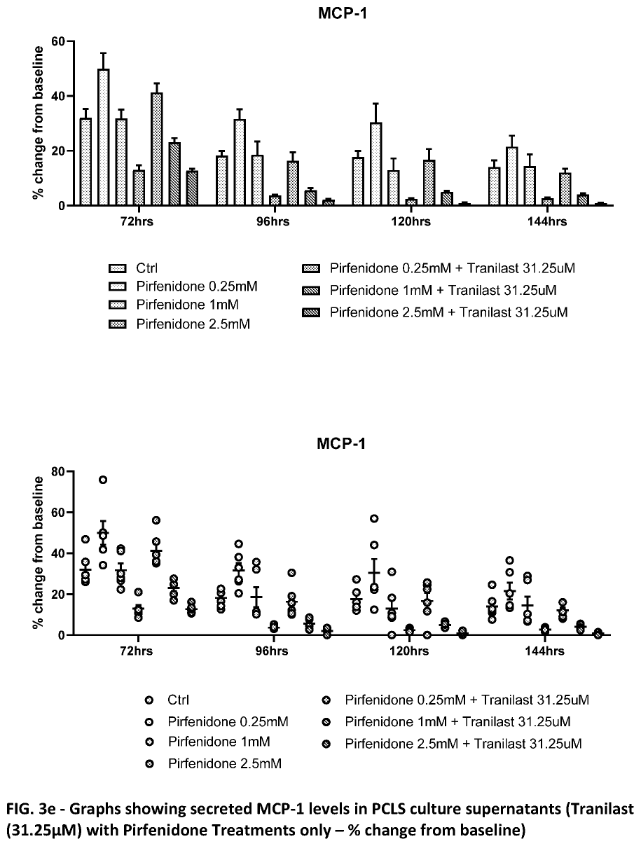

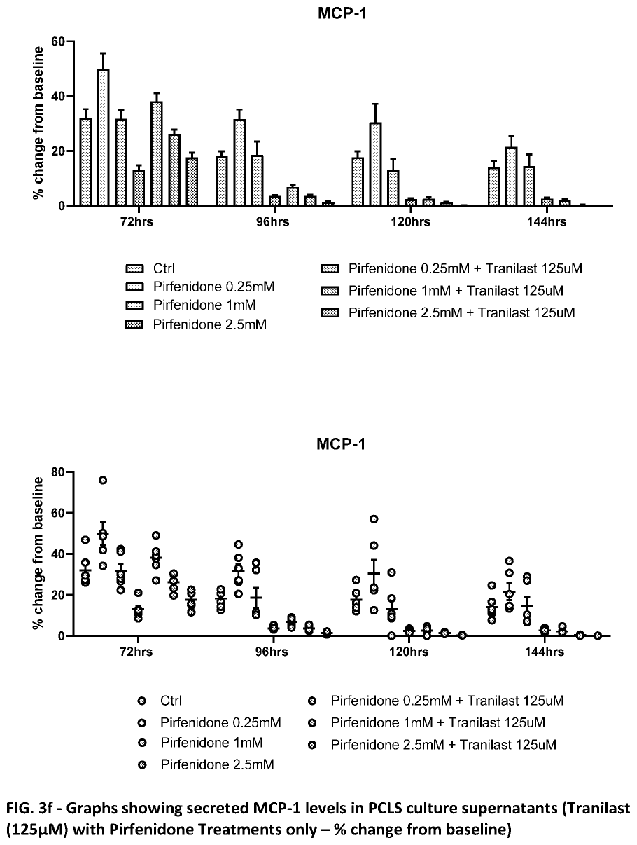

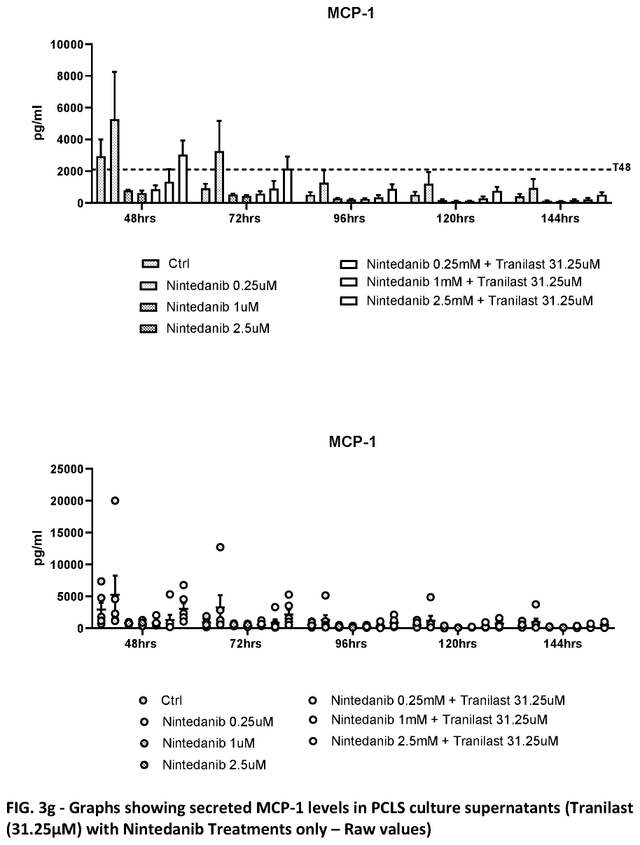

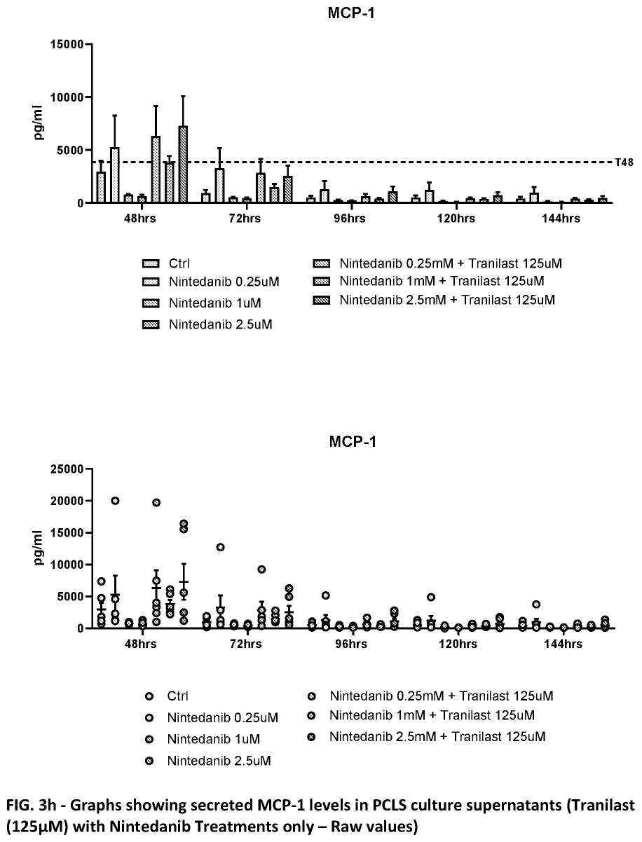

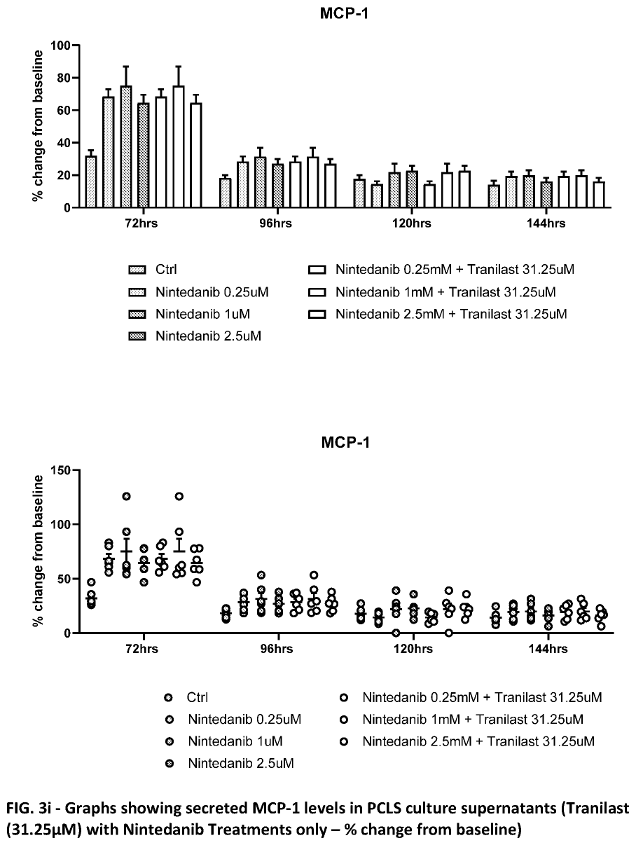

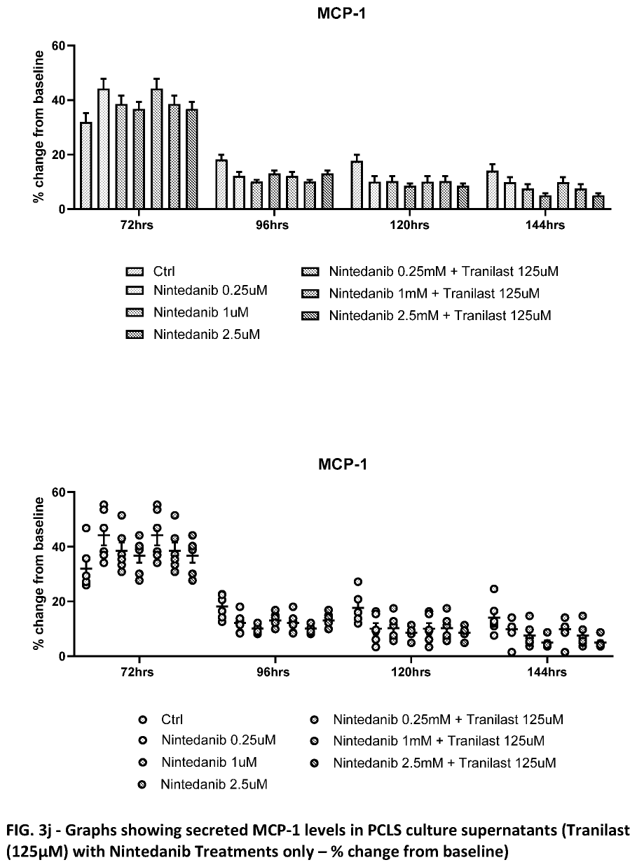

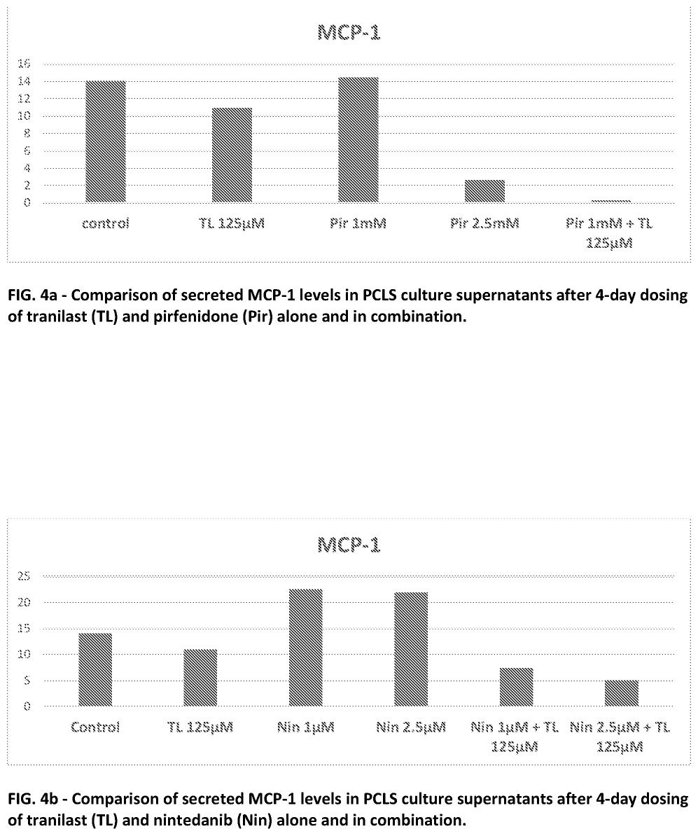

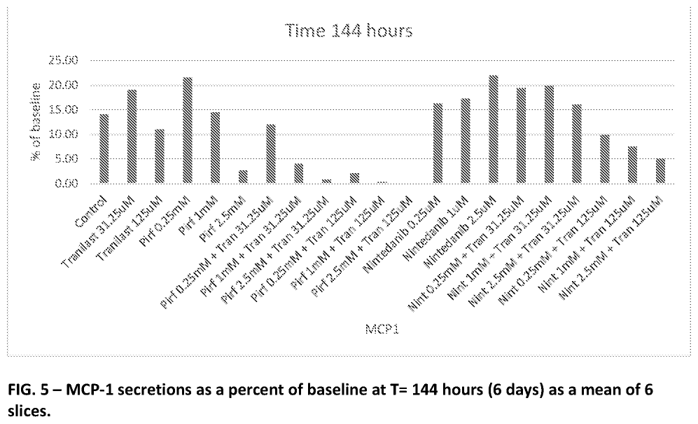

The results for the inflammation marker MCP-1 are shown in FIGS. 3a-3j, FIGS. 4a and 4b, and FIG. 5. Graphs are represented by n=6 tissue culture media per condition, per time point. Data is presented as mean±SEM with individual values per tissue culture media. MCP-1 secretion was concentration dependently attenuated by pirfenidone alone, although little to no effect was seen with nintedanib at any concentration tested (FIG. 3a). High concentration Tranilast marginally reduced MCP-1 levels although the effect was of a lower magnitude to that obtained with high concentration pirfenidone (FIG. 3b). Co-treatment with tranilast and pirfenidone at both concentrations (31.25 μM and 125 μM, FIGS. 3c and 3d) further attenuated MCP-1 secretion compared to individual treatments alone, with the most potent effect seen with high concentration Tranilast, FIGS. 3e, 3f, 3g, and 3h. Co-treatment with tranilast and nintedanib at both concentrations (31.25 μM and 125 μM) had little to no effect on MCP-1 secretion, FIGS. 3i and 3j. FIG. 4a shows that after 4-day dosing high concentration pirfenidone (Pir) strongly inhibited MCP-1 whereas suboptimal concentrations of tranilast (TL) or pirfenidone alone had minimal effect. Combined suboptimal concentrations of tranilast and pirfenidone completely ablated MCP-1 secretion and were superior to high concentration pirfenidone. FIG. 4b shows that after 4-day dosing nintedanib (Nin) alone had no effect on MCP-1 whereas suboptimal concentrations of tranilast (TL) combined with an ineffective concentration of nintedanib had a strong effect on MCP-1. FIG. 5 shows the effect MCP-1 secretions as a percentage of baseline (mean of 6 slices) at 6 day dosing of tranilast (Tran) alone, pirfenidone (Pir) alone, nintedanib (Nin) alone, combinations of tranilast and pirfenidone, and combinations of tranilast and nintedanib against control.

Little to no effect on IL-6 secretion was seen with pirfenidone, nintedanib or Tranilast at any concentration tested. Co-treatment with tranilast and pirfenidone at both concentrations (31.25 μM and 125 μM) attenuated IL-6 secretion in combination only with the most potent effect seen with high concentration Tranilast. Co-treatment with tranilast and nintedanib at both concentrations (31.25 μM and 125 μM) had little to no effect on MCP-1 secretion.

Levels of GM-CSF and IL-1β in the PCLS culture media was extremely low, approaching the levels of detection on the MSD assay used to quantify expression and therefore it is difficult to draw meaningful conclusion from these data.

IL-8 secretion was concentration dependently attenuated by pirfenidone alone, although little to no effect was seen with nintedanib or Tranilast at any concentration tested. Co-treatment with tranilast and pirfenidone at 31.25 μM had a further small attenuating effect on IL-8 secretion compared to individual treatments alone, however a more profound effect on IL-8 secretion was seen in PCLS co-treatment with tranilast and pirfenidone at 125 μM. Co-treatment with tranilast and nintedanib at 31.25 μM had little to no effect on IL-8 secretion although a small attenuation of IL-8 secretion was observed in PCLS co-treated with tranilast and nintedanib at 1251 μM.

TIMP-1 secretion was very variable between PCLS and showed little to no attenuation with individual treatments. However, co-treatment with tranilast and pirfenidone at both concentrations (31.25 μM and 125 μM) attenuated TIMP-1 secretion in combination only with the most potent effect seen with high concentration Tranilast. Similarly, co-treatment with tranilast and nintedanib at both concentrations (31.25 μM and 125 μM) attenuated TIMP-1 secretion in combination only with the most potent effect seen with high concentration Tranilast.

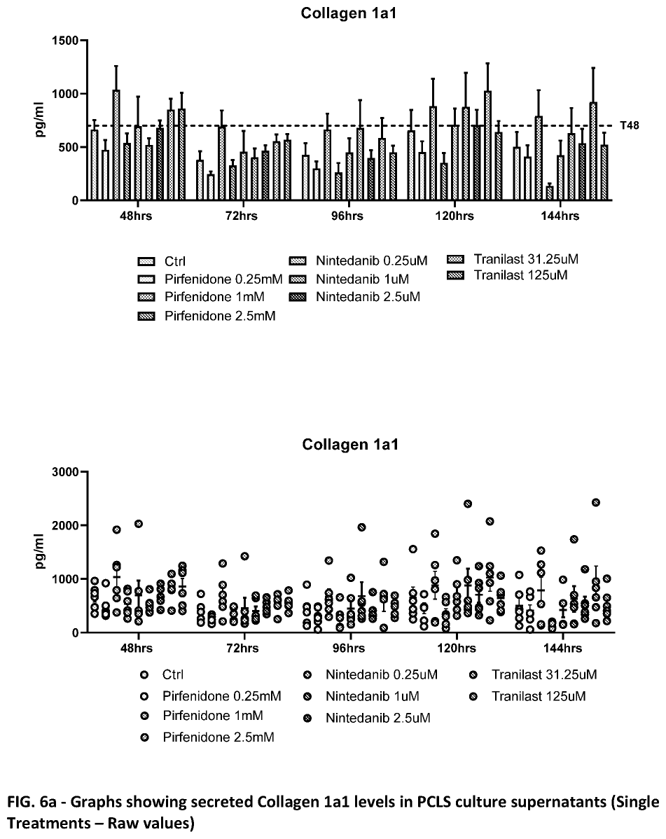

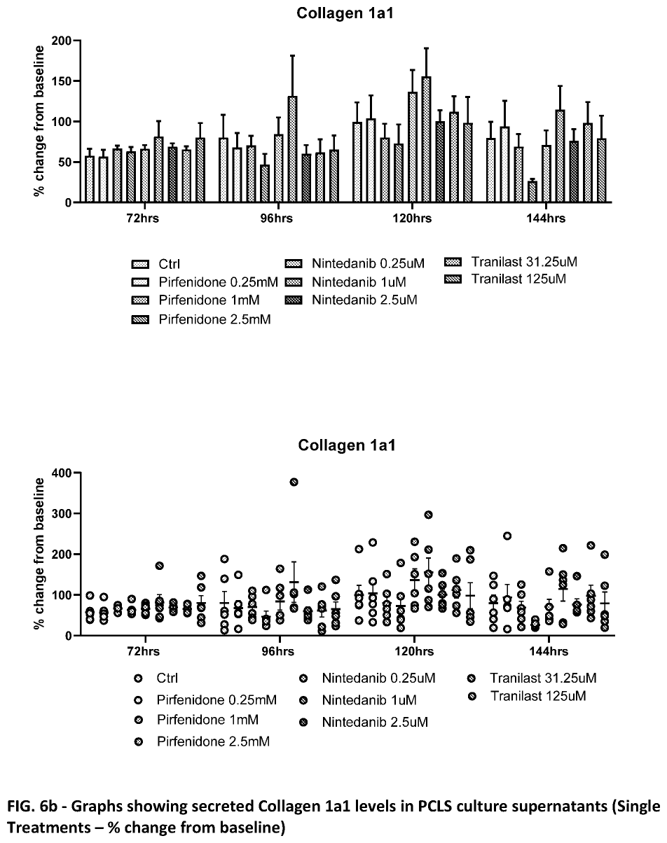

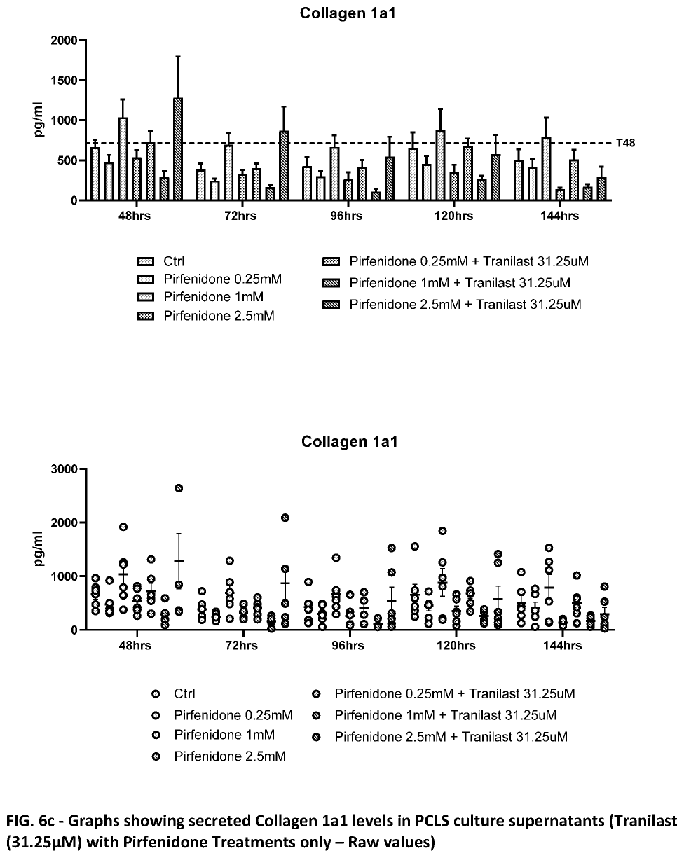

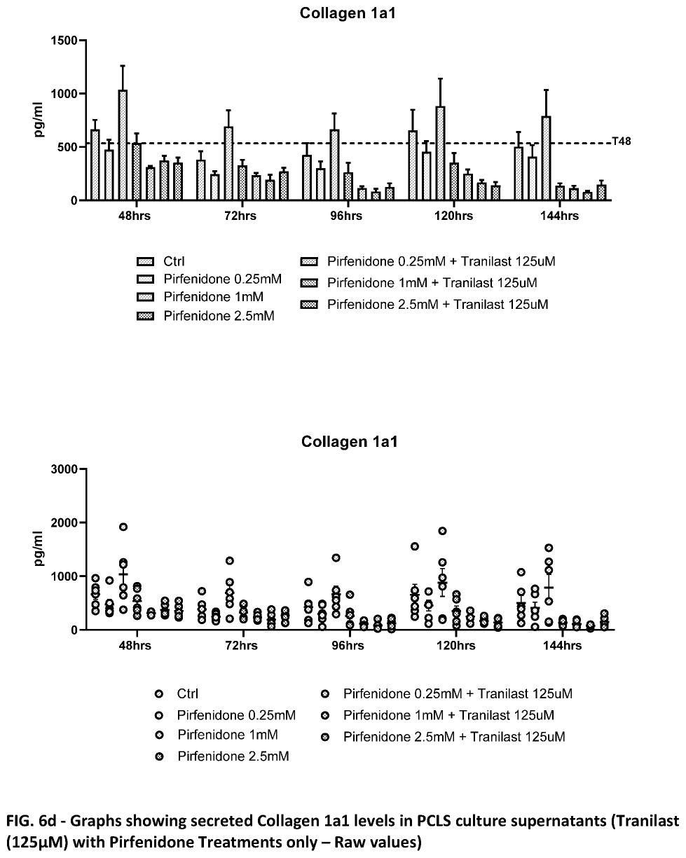

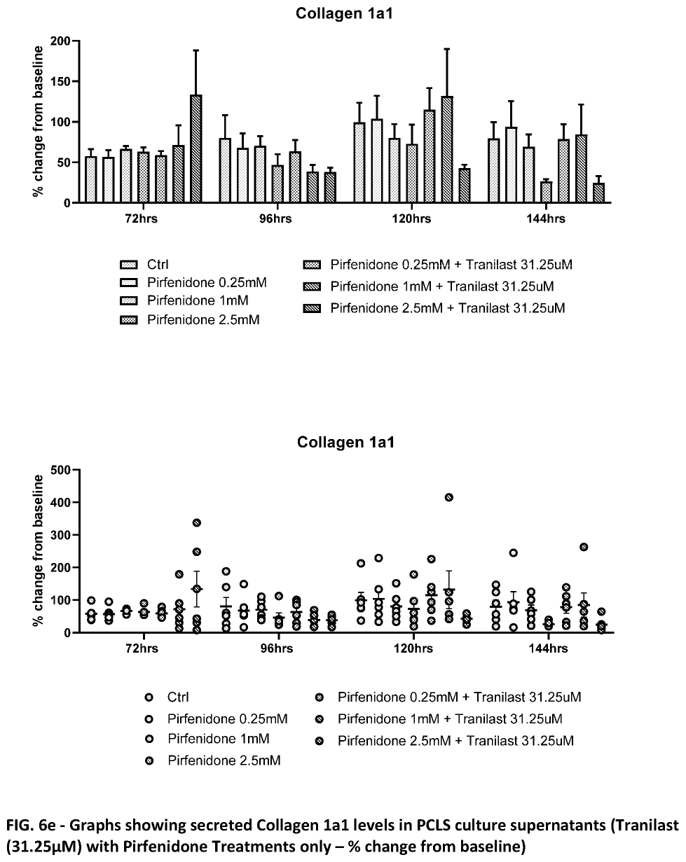

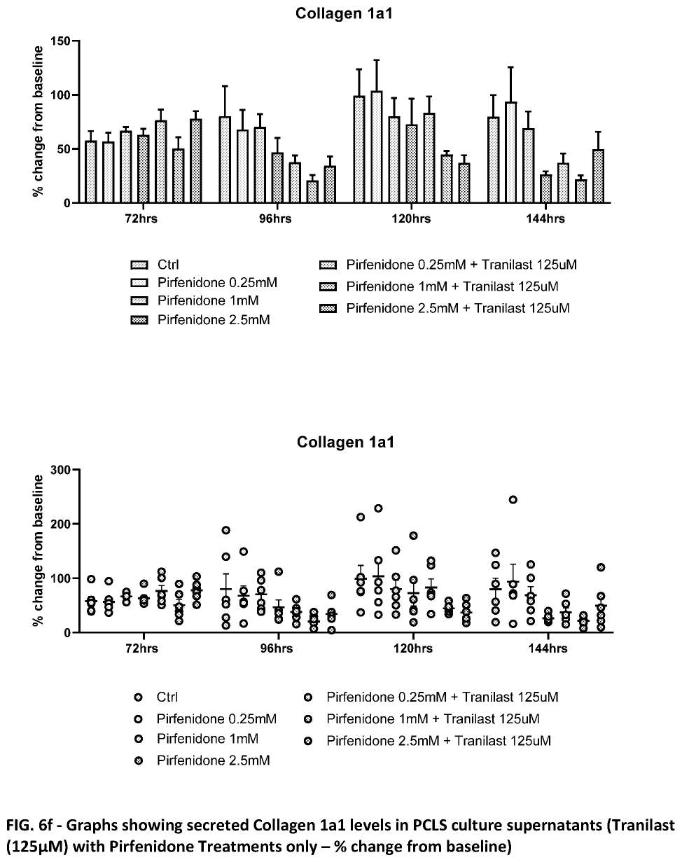

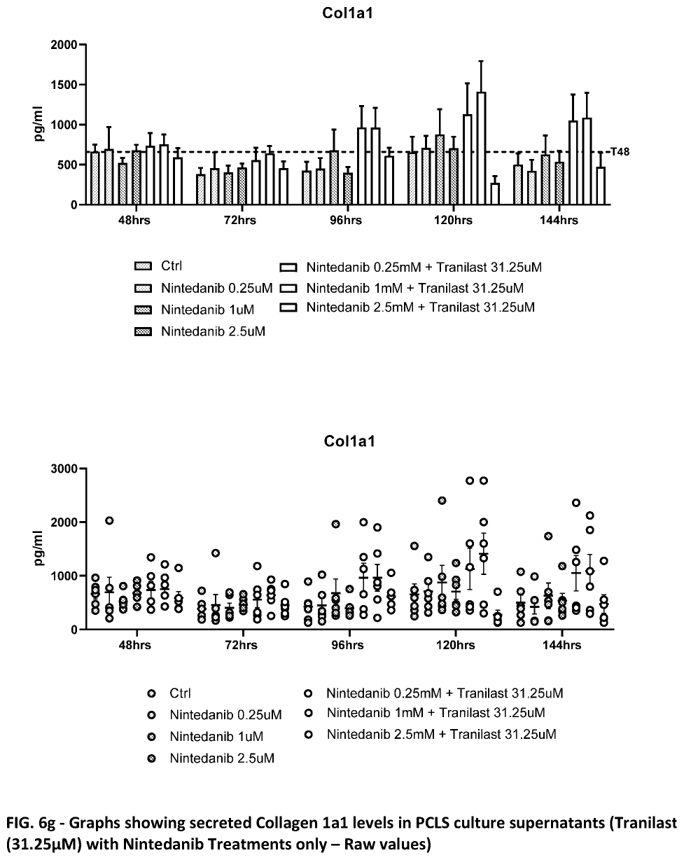

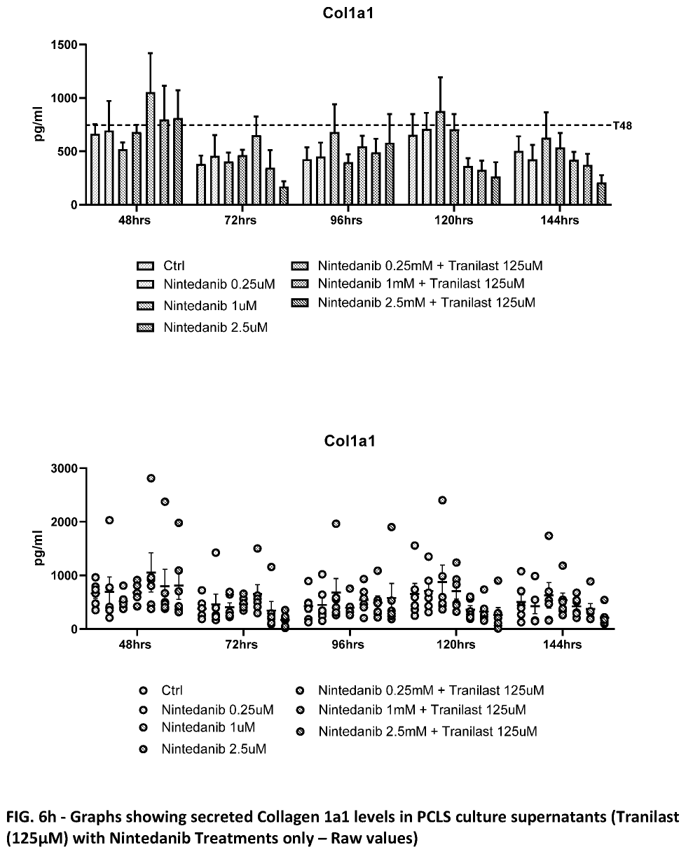

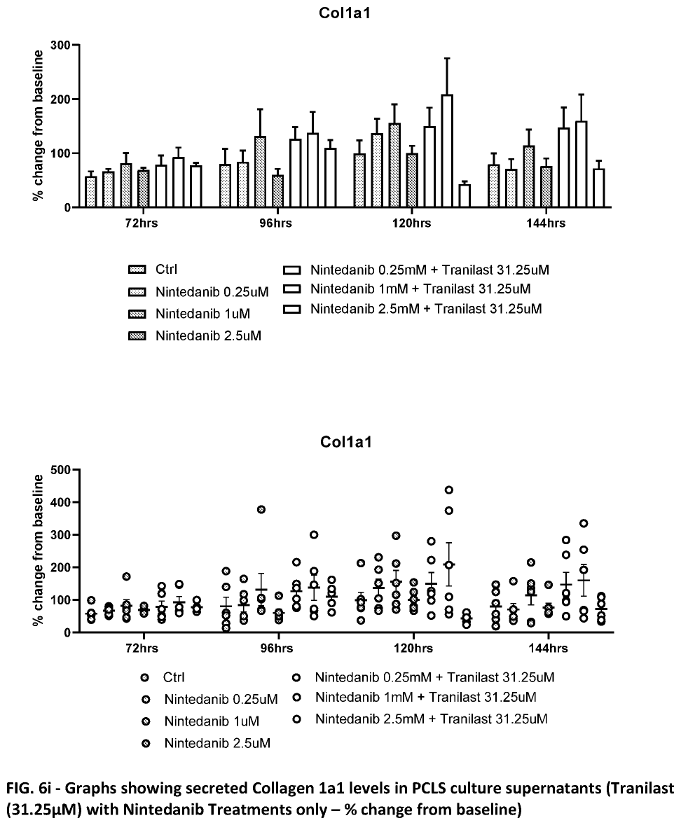

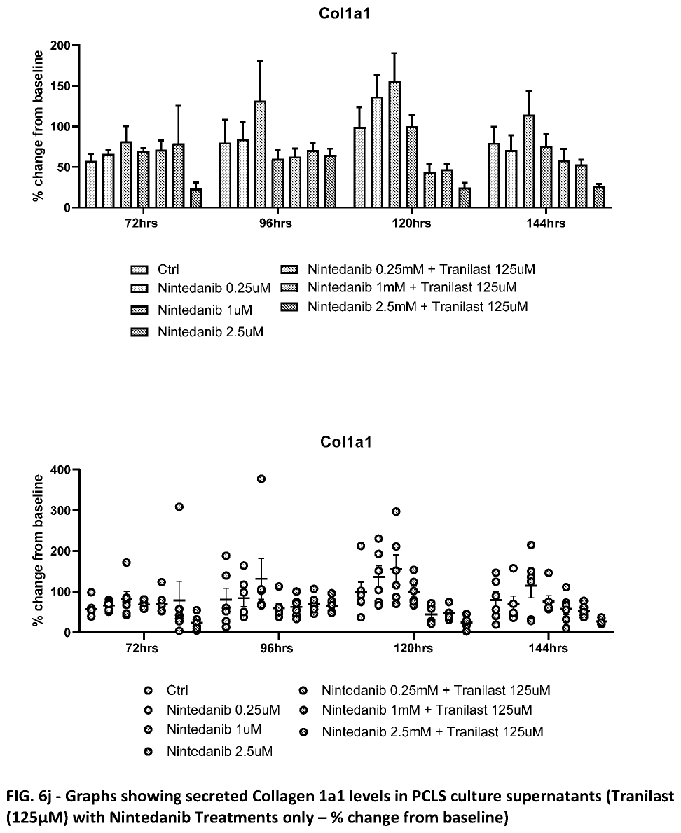

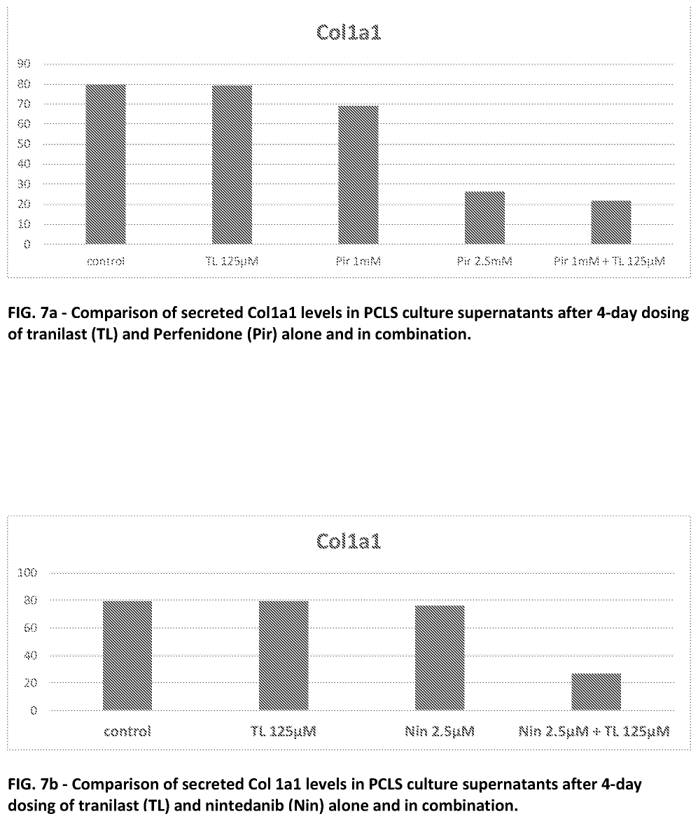

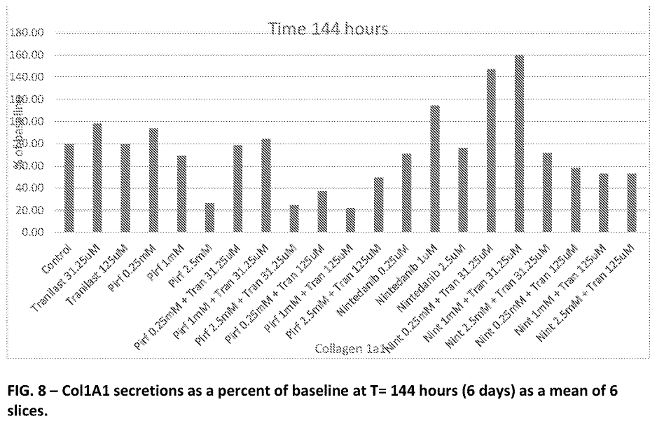

The results for the fibrotic marker collagen 1a1 (Col1A1) are shown in FIGS. 6a-6j, FIGS. 7a and 7b, and FIG. 5. Graphs are represented by n=6 tissue culture media per condition, per time point. Data is presented as mean±SEM with individual values per tissue culture media. Collagen 1a1 secretion was concentration dependently attenuated by pirfenidone alone, although little to no effect was seen with nintedanib at any concentration tested, FIG. 6a. High concentration tranilast marginally reduced Collagen 1a1 secretion although the effect was of a lower magnitude to that obtained with high concentration pirfenidone, FIG. 6b. Co-treatment with tranilast and pirfenidone at 31.25 μM had a further small attenuating effect on collagen 1a1 secretion compared to individual treatments alone, FIG. 6c In contrast, PCLS co-treatment with tranilast and pirfenidone at 125 μM (FIG. 6d) further attenuated collagen 1a1 secretion compared to individual treatments alone, FIGS. 6e and 6f. Co-treatment with tranilast and nintedanib at 31.25 μM had little to no effect on collagen 1a1 secretion, FIG. 6g. FIG. 6h depicts the graphs showing secreted Collagen 1a1 levels in PCLS culture supernatants (Tranilast (125 μM) with nintedanib. However, while little to no effect was seen with nintedanib at any concentration tested in isolation, co-treatment with tranilast and nintedanib at 125 μM attenuated collagen 1a1 secretion in a concentration dependent manner, FIGS. 6i and 6j. FIG. 7a shows that after 4-day dosing high concentration pirfenidone (Pir) strongly inhibited Col1A1 whereas suboptimal concentrations of tranilast (TL) or pirfenidone alone had minimal effect. Combined suboptimal concentrations of tranilast and pirfenidone completely ablated MCP-1 secretion and were superior to high concentration pirfenidone. FIG. 7b shows that after 4-day dosing neither nintedanib (Nin) nor tranilast (TL) alone effected MCP-1 whereas suboptimal concentrations of tranilast (TL) combined with an ineffective concentration of nintedanib had a strong effect on Col1A1. FIG. 8 shows the effect Col1A1 secretions as a percentage of baseline (mean of 6 slices) at 6 day dosing of tranilast (Tran) alone, pirfenidone (Pir) alone, nintedanib (Nin) alone, combinations of tranilast and pirfenidone, and combinations of tranilast and nintedanib against control.

Fibronectin: Fibronectin secretion showed little to no attenuation with individual treatments except for an attenuation with high concentration pirfenidone at 144 hrs. Co-treatment with tranilast and pirfenidone at both concentrations (31.25 μM and 125 μM) attenuated Fibronectin secretion. Co-treatment with tranilast and nintedanib at both concentrations (31.25 μM and 125 μM) had little to no effect on Fibronectin secretion.

The claimed invention is:

1. A method of treating idiopathic pulmonary fibrosis (IPF) comprising, consisting essentially of, or consisting of the step of administering to a patient in need thereof a therapeutically effective amount of a combination of tranilast and pirfenidone, or a therapeutically effective amount of a combination of tranilast and nintedanib, to treat idiopathic pulmonary fibrosis (IPF).

2. The use of a therapeutically effective amount of a therapeutically effective amount of a combination of tranilast and pirfenidone, or of a therapeutically effective amount of a combination of tranilast and nintedanib, to treat idiopathic pulmonary fibrosis (IPF).

3. A pharmaceutical composition comprising, consisting essentially of, or consisting of a therapeutically effective amount of a combination of tranilast and pirfenidone, or a therapeutically effective amount of a combination of tranilast and nintedanib, to treat idiopathic pulmonary fibrosis (IPF) and a pharmaceutically acceptable excipient.

4. A method of claim 1 or the use of claim 2, wherein the tranilast is administered as an adjunctive therapeutic to the treatment of idiopathic pulmonary fibrosis (IPF) by administering pirfenidone or nintedanib.

5. The method of claim 1, the use of claim 2, or the pharmaceutical composition of claim 3, wherein the combination of (i) the tranilast and pirfenidone or (ii) the tranilast and nintedanib is synergistically effective to treat idiopathic pulmonary fibrosis (IPF).

6. The method of claim 1, the use of claim 2, or the pharmaceutical composition of claim 3, wherein the tranilast is a tranilast cocrystal or a tranilast salt.

7. The method of claim 1, the use of claim 2, or the pharmaceutical composition of claim 3, wherein the tranilast is a 1:1 tranilast I-lysine salt.

8. A method of treating idiopathic pulmonary fibrosis (IPF) comprising, consisting essentially of, or consisting of the step of administering to a patient in need thereof a therapeutically effective amount of a combination of the tranilast and pirfenidone to treat idiopathic pulmonary fibrosis (IPF).

9. The method of claim 8, wherein the tranilast is administered as an adjunctive therapeutic to the treatment of idiopathic pulmonary fibrosis (IPF) by administering pirfenidone.

10. The use of a therapeutically effective amount of a combination of the tranilast and pirfenidone to treat idiopathic pulmonary fibrosis (IPF).

11. A pharmaceutical composition comprising, consisting essentially of, or consisting of a therapeutically effective amount of a combination of the tranilast and pirfenidone to treat idiopathic pulmonary fibrosis (IPF) and a pharmaceutically acceptable excipient.

12. The method of claim 8 or 9, the use of claim 10, or the pharmaceutical composition of claim 11, wherein the combination of the tranilast and pirfenidone is synergistically effective to treat idiopathic pulmonary fibrosis (IPF).

13. The method of claim 8 or 9, the use of claim 10, or the pharmaceutical composition of claim 11, wherein the tranilast is a tranilast cocrystal or a tranilast salt.

14. The method of claim 8 or 9, the use of claim 10, or the pharmaceutical composition of claim 11, wherein the tranilast is a 1:1 tranilast I-lysine salt.

15. A method of treating idiopathic pulmonary fibrosis (IPF) comprising, consisting essentially of, or consisting of the step of administering to a patient in need thereof a therapeutically effective amount of a combination of the tranilast and nintedanib to treat idiopathic pulmonary fibrosis (IPF).

16. The method of claim 15, wherein the tranilast is administered as an adjunctive therapeutic to the treatment of idiopathic pulmonary fibrosis (IPF) by administering nintedanib.

17. The use of a therapeutically effective amount of a combination of the tranilast and nintedanib to treat idiopathic pulmonary fibrosis (IPF).

18. A pharmaceutical composition comprising, consisting essentially of, or consisting of a therapeutically effective amount of a combination of the tranilast and nintedanib to treat idiopathic pulmonary fibrosis (IPF) and a pharmaceutically acceptable excipient.

19. The method of claim 15 or 16, the use of claim 17, or the pharmaceutical composition of claim 18, wherein the combination of the tranilast and pirfenidone is synergistically effective to treat idiopathic pulmonary fibrosis (IPF).

20. The method of claim 15 or 16, the use of claim 17, or the pharmaceutical composition of claim 18, wherein the tranilast is a tranilast cocrystal or a tranilast salt.

21. The method of claim 15 or 16, the use of claim 17, or the pharmaceutical composition of claim 18, wherein the tranilast is a 1:1 tranilast I-lysine salt.

22. The method of any one of claims 1, 4-9, 12-16, and 19-21, wherein the tranilast is administered as an inhalable formulation.

23. The method of claim 22, wherein pirfenidone or nintedanib is administered as an oral pharmaceutical composition.
